SEQUENCE LISTING (2 sequences)

SEQ ID NO: 1 - RNA, 21 nt, Artificial Sequence
  Synthetic oligonucleotide
aaggagcuggauggcuggguu

SEQ ID NO: 2 - RNA, 21 nt, Artificial Sequence
  Synthetic oligonucleotide
aacguacgcggaauacaacga

(19) United States
(12) Patent Application Publication
(10) Pub. No.: US 2006/0051819 A1
(43) Pub. Date: Mar. 9, 2006

(54) Detection, evaluation and treatment for advanced prostate cancer

(75) Inventors: Milton Whitaker Datta, Atlanta, GA (US); Sumana Datta, Decatur, GA (US); Ariel Ruiz Altaba, New York, NY (US)

Correspondence Address: CHALKER FLORES, LLP, 2711 LBJ FRWY, Suite 1036, DALLAS, TX, 75234, US

(73) Assignee: The Texas A&M University System, College Station, TX (US)

(21) Appl. No.: 11/156,879

(22) Filed: Jun. 17, 2005

Related U.S. Application Data
(60) Provisional application No. 60/580,453, filed on Jun. 17, 2004.

(51) Int. Cl. G01N 33/574; A61K 48/00

(52) U.S. Cl. 435/7.23; 514/44

(57) ABSTRACT

The present invention includes novel compositions, methods and systems for detecting, evaluating and inhibiting the proliferation of prostate cancer cells by detecting and inhibiting the expression and activity of the proteoglycan Perlecan.

### TECHNICAL FIELD OF THE INVENTION

The present invention relates in general to the field of prostate cancer diagnosis and treatment, and more particularly, to methods and compositions for detecting, evaluating and inhibiting the proliferation of prostate cancer cells.

### BACKGROUND OF THE INVENTION

This application claims priority to U.S. Provisional Patent Application Ser. No. 60/580,453, filed Jun. 17, 2004. The U.S. Government may own certain rights in this invention pursuant to the terms of NIH grants 5R01CA078736-04 and 1R01NS037352-01. Without limiting the scope of the invention, its background is described in connection with the prostate gland. The prostate gland is located between the bladder and the rectum and wraps around the urethra. Composed of glandular tissue that produces a milky fluid and smooth muscles, the prostate contracts during sex and produces fluid into the urethra where it mixes with other fluid and sperm to form semen. The prostate gland converts testosterone to a more powerful male hormone, dihydrotestosterone, which affects the size of the gland and plays an important role in prostate cancer.

Malignant tumors that arise in the prostate gland may eventually spread through the blood and lymph fluid to other organs, bones and tissues. While prostate cancer is the most commonly diagnosed cancer in the United States, it is also the second leading cause of cancer death in American men after non-melanoma skin cancer. Although prostate cancer is common in other industrialized nations, e.g., Japan, death rates from prostate cancer are significantly lower in Japan than in the United States. However, studies show that Japanese men that migrate to the United States die of prostate cancer with increasing frequency as a function of the number of years they reside in the United States. One very likely explanation is changes in diet that affect disease susceptibility and progression.

Presently, physicians detect cancers by finding a lump in the prostate gland, e.g., during a routine checkup or an examination prompted by a patient's complaint of sudden urinary discomfort or occasional impotence. In some instances, prostate cancer is detected in the course of treatment for a disorder called benign prostatic hyperplasia (BPH). BPG is an aging-related enlargement of the prostate that affects more than half of all men older than 45 and causes urinary troubles similar to those caused by a prostate tumor. When BPG symptoms become too troublesome, a transurethral resection of the prostate may be performed and the excised tissue is analyzed under a microscope for evidence of malignancy, which is occasionally found.

Another method for detecting prostate cancer is a simple blood test for prostate specific antigen (PSA). Increased PSA levels may signal the presence of prostate cancer in individuals who have not yet displayed symptoms of prostate abnormalities. However, problems remain with the number of false positives, and more importantly, false negatives in PSA evaluations. What are needed are one or more improved molecular markers for prostate cancer identification and disease progression to identify patients who are at risk for aggressive disease and would benefit from early treatment. Also needed are novel therapies for prostate cancer treatment.

### SUMMARY OF THE INVENTION

The present invention provides novel tools for the clinical detection and evaluation of prostate cancer cells. The compositions, methods and systems disclosed herein find particular usefulness in the diagnosis of advanced prostate cancer without the need for new equipment or extensive training of laboratory personnel. The present inventors have recognized that the secreted proteoglycan Perlecan may be used as a marker for the testing of fluid samples due to their up-regulation in prostate cancer cells.

More particularly, the present invention includes a method of diagnosing cancer pathology in a subject including the steps of obtaining tissue sample from the subject; and analyzing the level of expression of Perlecan, Patched or Smoothened, for elevated Perlecan, Patched or Smoothened expression indicative of cancer or pre-cancer. The tissue sample may be, e.g., a prostate sample that may also be suspected of prostatitis or prostate cancer, a prostate cancer exudates, urine, a tissue sample is stained with haematoxylin and eosin or even a serum or semen sample. The analysis may be performed by light microscopy, e.g., using immunostaining or electron microscopy.

Another example of the invention is a method of treating an individual with prostate cancer or at risk of developing prostate cancer, by identifying an individual with prostate cancer or at risk of developing prostate cancer, administering a dose of a Perlecan specific siRNA to the individual in an amount that is effective to inhibit the expression of Perlecan and monitoring the expression of Perlecan in the individual, wherein inhibiting the expression of Perlecan inhibits the proliferation of prostate cancer cells, thereby treating the individual. The composition may be administrates in an oral, transdermal, intravenous, intraperitoneal, pulmonary, rectal, ocular, subcutaneous, intramuscular, transdermal, topical, intraosseal, epidural, dural, intranasal, and implanted formulation. The formulation may be a dose packed into a vial, capsule, caplet, softgel, gelcap, suppository, film, granule, gum, insert, pastille, pellet, troche, lozenge, disk, poultice or wafer. For example, the dose may be from about 5 mg/kg to about 25 mg/kg and the individual may be a human.

Yet another aspect of the present invention is a method of reducing the risk of recurrence of prostate cancer in an individual, wherein said individual previously had been treated for prostate cancer by administering a dose of one or more Perlecan specific siRNA to the individual in an amount that is effective to inhibit the expression of Perlecan. The step of periodically providing one or more Perlecan specific siRNA to the individual may also include providing the individual with an amount of Perlecan specific siRNA that is effective to inhibit the expression of Perlecan, thereby reducing the risk of recurrence of prostate cancer in the individual. The method may also include the step of monitoring the level of expression of Perlecan in the individual.

In yet another aspect of the present invention, a system for identifying inhibitors of Perlecan may include adding one or more agents or molecules suspected of modifying Perlecan expression to a cell that expressed Perlecan and analyzing the level of expression of Perlecan in the cells, for an elevated level of expression indicative of cancer or pre-cancer. The level of expression of Prelecan in the cells is compared to a non-treated cell control and modified levels identified, e.g., lowered or elevated. The one or more agents may be selected from nucleic acids, peptides, small molecules, proteins, lipids, carbohydrates and combinations thereof. In one example, the level of Perlecan expression is determined via a high-throughput, automated robot. Cells that may be evaluated include, e.g., prostate cancer, small cell lung cancer, stomach cancer, pancreatic cancer, malignant melanoma, basal cell carcinoma, colon cancer and glioma cells.

The present invention also includes a pharmaceutical composition that includes a pharmaceutically effective amount of a Perlecan specific siRNA, wherein the Perlecan specific siRNA has (SEQ ID NO.: 1). The composition may further include one or more carriers. For example, the composition may include Perlecan specific siRNA that is packaged into a vial, capsule, caplet, softgel, gelcap, suppository, film, granule, gum, insert, pastille, pellet, troche, lozenge, disk, poultice or wafer and may be in solid or liquid form, dry or resuspended and the like. One example of a pharmaceutically effective amount is from about 5 mg/kg to about 25 mg/kg, e.g., of a Perlecan specific siRNA having SEQ ID NO.: 1.

More particularly, the present invention takes advantage of a heretofore unknown mechanism for the detection and clinical evaluation of advanced prostate cancer. It has been found that Perlecan and Patched are integral to the human Hedgehog (hH) signaling pathway and that their expression is up-regulated in advanced prostate cancer samples. Therefore, the level of expression and localization of Perlecan and Patched are markers for advanced prostate cancer versus normal or pre-cancerous lesion high grade prostatic intraepithelial neoplasia (HGPIN) samples. In fact, the present inventors have found high secreted levels of Perlecan protein in cases of advanced prostate cancer, which are not identifiable using current techniques. To improve the diagnosis and the determination of the level of disease progression tissue samples the expression level of Perlecan and/or Patched may be determined from, e.g., a biopsy, a serum or a semen samples, and the profile compared to normal samples and samples of cell lines and primary cell cultures from prostate cancer cells in different levels of disease progression to determine the disease progression of the patient. It has been found that Perlecan is secreted at high levels into the lumen of diseased prostate glands, as such, the present invention provides a non-invasive test for advanced prostate cancer by testing for the level of Perlecan in a patient sample.

The method of detection includes the determination of expression of the proteoglycan Perlecan or the human homolog to the Drosophila Patched protein, e.g., human Perlecan and/or human Patched, using antibodies (poly or monoclonal), nucleic acid probes, PCR and the like that are specific to Perlecan and/or Patched to determine their level of expression. The detection of high levels of Perlecan and/or Patched from a biological sample or a supernatant, e.g., serum or semen samples and/or cell culture, are indicative of advanced prostate cancer.

Perlecan may also be targeted in patients suspected of having a cancer by drugs that affect Perlecan and other proteins in the human Hedgehog (hH) signaling pathway, e.g., small cell lung cancer, stomach cancer, pancreatic cancer and gliomas. Inhibition of the hH pathway downstream from hH signal transduction, e.g., at Perlecan, Smoothened and the like may be used to decrease growth of human prostate cancer cells. Also, specific and non-specific inhibitors of the downstream elements of the hH pathway by inactivation of the transcription, translation, and/or activity of Perlecan and Smoothened may be of therapeutic value to slow the growth of advanced prostate cancer cells and/or trigger programmed cell death (apoptosis) of the advanced prostate cancer cells.

One such molecule that has been studies is RNAi specific to Perlecan, which led to decreased Perlecan expression and BrdU incorporation in prostate cancer cell lines. As such, the methods of the present invention may be used to identify, characterize, develop and detect small molecule inhibitors that will specifically target Perlecan and/or Patched and lead to effective therapies for advanced prostate cancer. For example, an RNAi sequence 5′ AAGGAGCUGGAUGGCUGGGUU 3′ (SEQ ID NO.: 1) was shown to decrease BrdU incorporation in both the androgen sensitive cell line LNCaP (up to about 60%) and the androgen insensitive cell line PC3 (about 10%). As the skilled artisan will appreciate, and as is available from companies that have algorithms and expertise in the area of RNAi optimization, e.g., Dharmacon, optimized RNAi constructs and/or small molecule, peptide, protein or protein-based therapeutics may be developed that specifically target Perlecan and/or Smoothened expression in cancer cells. Furthermore, Perlecan may also be a marker for other human Hedgehog (Hh) associated signaling related cancers, for some of which there is presently no available reliable method of detection, e.g., small cell lung cancer, stomach cancer, pancreatic cancer, malignant melanoma, colon cancer and gliomas. The present inventors have also found that Perlecan message levels are increased in these other types of cancers, therefore, Perlecan expression levels may be routinely evaluated at the nucleic acid and/or protein level in patients in a non-invasive and inexpensive manner.

The present invention provides an alternative method of non-invasive detection for prostate cancer as compared with Prostate Specific Antigen (PSA). The PSA assay is the subject of continued scrutiny as the current level of PSA expression that is considered to be positive for prostate cancer misses around 50% of the prostate cancer occurrences. However, lowering the positive level of PSA also increases dramatically the number of false positive readings. Next, patients may undergo invasive histological tests and evaluation, however, these tests are also subject to variations in interpretation depending on the level of experience and training of the pathologist, because the Gleason Scores vary with morphological characteristics and the stage of the disease. Unfortunately, if a patient is diagnosed with an advanced prostate cancer there are few, if any, treatments that provide a survival advantage.

The present invention provides a method and system for not only detecting and evaluating the status of prostate cancer, but also for the isolation, characterization and evaluation of small molecule, antibody, nucleic acid and other candidates for therapies against prostate cancer and for other cancers, including, small cell lung cancer, stomach cancer, pancreatic cancer, malignant melanoma, basal cell carcinoma, colon cancer and gliomas. The present invention may be used to conclusively evaluate candidates that decrease the activity of Perlecan, Smoothened and Gli-I to decrease the growth or eliminate prostate and other cancers of the hH pathway.

### BRIEF DESCRIPTION OF THE DRAWINGS

For a more complete understanding of the features and advantages of the present invention, reference is now made to the detailed description of the invention along with the accompanying figures and in which:

FIGS. 1A to 1D show the histology of Perlecan expression in normal prostate tissue and prostate tumors;

FIG. 1E is a western blot showing Perlecan expression in normal and tumor cells;

FIG. 1F is a graphical representation of the regions of Perlecan;

FIG. 2A is a graph of Perlecan expression by real-time PCR;

FIG. 2B is a western blot that shows protein expression in the analyzed tissue;

FIG. 2C is a graph that shows the effect of Perlecan siRNA on BrdU incorporation;

FIG. 3A is an image showing the co-immunoprecipitation of SHH with an anti-Perlecan antibody probed with anti-SHH; and

FIG. 3B is a graph that compares the expression of Perlecan (black columns), PTCH (striped columns) and GLI1 (grey columns) normalized to β-ACTIN levels in LNCaP cells treated with Perlecan siRNA and controls.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, “Perlecan” is used to describe a single copy gene in humans that includes, 94 exons with the predicted core protein sequence having a molecular mass of 467 kilodaltons. The nucleic acid sequence that encodes mouse perlecan encodes a protein core with a molecular mass of approximately 396 kilodaltons. Perlecan, for all purposes of the present invention, may also be referred to as a basement membrane heparan sulfate PG, and/or a heparan sulfate PG of basement membranes. Domain I of perlecan is encoded by 5 exons (numbered 2 to 6) and is postulated to contain three heparan sulfate GAG attachment sites and is unique to Perlecan showing no homology to other known sequences. The locations of the three Ser-Gly consensus heparan sulfate GAG attachment sites at the N-terminus correspond with the number and position of known GAG chains. Domain II is related to the LDL binding domain present in the LDL-receptor, whereas Domain III shares some homology to the globule-rod regions of the laminin short arms. Domain IV is highly repetitive with numerous immunoglobulin-like repeats with highest similarity to those of neural cell adhesion molecule (N-CAM). Domain V has three globular repeats very similar to the domain G repeats in the laminin A chain and the equivalent segment of the A chain homolog, merosin, and two epidermal growth factor-like regions. Certain exons of Perlecan may also be differentially spliced (see U.S. Pat. No. 6,432,636, relevant portions incorporated herein by reference).

The present invention includes a method of diagnosing cancer pathology in which a tissue sample from the subject is analyzed for the aberrant expression of Perlecan, Patched and/or Smoothened. As used herein the term “expression indicative of cancer or pre-cancer” is used to describe an increase in the expression, message life, protein life, gene and/or protein expression and/or degradation that is greater than normal levels of expression. For example, the level of expression of Perlecan, Patched and/or Smoothened will be above that of normal/wild-type expression for a particular type of cell, such as a neuronal, prostate or other cell type. Expression levels of greater that 0.1, 1, 2, 5, 10, 15, 25 or greater percent may be indicative and/or diagnostic of the existence of cancer or pre-cancer.

As used herein, “amino acid sequence” refers to an amino acid sequence of a naturally occurring protein molecule, “amino acid sequence” and like terms, such as “polypeptide” or “protein” and are not meant to limit the amino acid sequence to the complete, native amino acid sequence associated with the recited protein molecule.

The term “antibody”, as used herein, is intended to refer to immunoglobulin molecules comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “antigen-binding portion” of an antibody (or simply “antibody portion”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′).sub.2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426: and Huston et al. (1988) Proc. Natl. Acad. Sci. USA 85:5879-5883). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123).

The present invention encompasses polyclonal, as well as monoclonal antibodies. The antibodies used in the methods of the invention may be prepared using various immunogens. The immunogen may be a human protein or subunit (e.g., any of the amino acid sequences set forth herein used as an immunogen) to generate antibodies that recognize human proteins. Such antibodies include, but are not limited to polyclonal, monoclonal, chimeric, single chain, Fab fragments, and an Fab expression library.

Various procedures known in the art may be used for the production of polyclonal antibodies to proteins and subunits. For the production of antibodies, various host animals can be immunized by injection with the peptide corresponding to the protein epitope of interest, including but not limited to rabbits, mice, rats, sheep, goats, etc. The peptide may be conjugated to an immunogenic carrier (e.g., diphtheria toxoid, bovine serum albumin (BSA) or keyhole limpet hemocyanin [KLH]). Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies directed toward proteins, any technique that provides for the production of antibody molecules by continuous cell lines in culture may be used (See e.g., Harlow and Line, Antibodies: A Laboratory Manual, Cold Spring Harbor Laboratory Press, Cold Spring Harbor, N.Y.). These include but are not limited to the hybridoma technique originally developed by Kohler and Milstein (Kohier and Milstein, Nature 256:495-497 [1975]), as well as the trioma technique, the human B-cell hybridoma technique (See e.g., Kozbor et al., Immunol. Today 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]).

In an additional embodiment of the invention, monoclonal antibodies can be produced in germ-free animals utilizing recent technology (See e.g., PCT/US90/02545). According to the invention, human antibodies may be used and can be obtained by using human hybridomas (Cote et al., Proc. Natl. Acad. Sci. U.S.A. 80:2026-2030 [198′)]) or by transforming human B cells with EBV virus in vitro (Cole et al, in Monoclonal Antibodies and Cancer Therapy, Alan R. Liss, pp. 77-96 [1985]).

“Nucleic acid sequence” as used herein refers to an oligonucleotide, nucleotide, or polynucteotide, and fragments or portions thereof, and to DNA or RNA of genomic or synthetic origin which may be single- or double-stranded, and represent the sense or antisense strand. Similarly, “amino acid sequence” as used herein refers to an oligopeptide, peptide, polypeptide, or protein sequence, and fragments or portions thereof, and to naturally occurring or synthetic molecules. As used herein the terms “protein,” “peptide” and “polypeptide” refer to compounds comprising amino acids joined via peptide bonds and are used interchangeably.

Nucleic acid sequence are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end oligonucleotides referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. In either a linear or circular DNA or RNA molecule, discrete elements are referred to as being “upstream” or 5′ of the “downstream” or 3′ elements. This terminology reflects the fact that transcription proceeds in a 5′ to 3′ fashion along the DNA or RNA strand. The promoter and enhancer elements which direct transcription of a linked gene are generally located 5′ or upstream of the coding region. However, enhancer elements can exert their effect even when located 3′ of the promoter element and the coding region. Transcription termination and polyadenylation signals are located 3′ or downstream of the coding region.

With PCR, it is possible to amplify a single copy of a specific target sequence to a level detectable by several different methodologies (e.g., hybridization with a labeled probe; incorporation of biotinylated primers followed by avidin-enzyme conjugate detection, incorporation ³²P-labeled deoxynucleotide triphosphates, such as DCTP or DATP, into the amplified segment). In addition to genomic DNA, any oligonucleotide sequence can be amplified with the appropriate set of primer molecules. In particular the amplified segments created by the PCR process itself are, themselves, efficient templates for subsequent PCR amplifications.

“Amplification” is a special case of nucleic acid replication involving template specificity. It is to be contrasted with nonspecific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (D. L. Kacian et al, Proc. Natl. Acad. Sci. USA 69:3038 [1972]). Other nucleic acid will not be replicated by this amplification enzyme. Similarly, in the case of T7 RNA polymerase, this amplification enzyme has a stringent specificity for its own promoters (M. Chamberlin et al Nature 228:227 [1970]). In the case of T4 DNA ligase, the enzyme will not ligate the two oligonucleotides where there is a mismatch between the oligonucleotide substrate and the template at the ligation junction (D. Y. Wu and R B. Wallace, Genomics 4:560 [1989]). Finally, Taq and Pfu polymerases by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “therapeutically effective amount” is meant an amount of the present invention effective to yield a desired therapeutic response. For example to prevent cancer or treat the symptoms of cancer in a host or an amount effective to treat cancer. The specific “therapeutically effective amount” will, obviously, vary with such factors as the particular condition being treated, the physical condition of the patient, the type of mammal being treated, the duration of the treatment, the nature of concurrent therapy (if any), and the specific formulations employed and the structure of the compounds or its derivatives.

The term “transfection” as used herein refers to the introduction of foreign DNA or RNA into cells. Transfection may be accomplished by a variety of means known to the art including calcium phosphate co-precipitation, DEAE-dextran-mediated transfection, polybrene-mediated transfection, electroporation, microillection, liposome fusion, lipofection, protoplast fusion, retroviral infection, and biolistics.

Familial prostate cancer studies have demonstrated a genetic link between prostate and brain tumors in a subset of families (1, 2), the linked susceptibility region (CArcinoma Brain Prostate, CABP) maps to human chromosome 1p36 (2). The present inventors identified HSPG2 (Perlecan) as a candidate gene for CABP due to its location at this genetic locus and its control of neural stem cell proliferation in Drosophila (3-5). Perlecan is an extracellular heparan sulfate proteoglycan known to bind a variety of growth factors (6). The present inventors have also linked Perlecan to signaling by FGF and Hedgehog (HH) in the Drosophila brain (4). Based on the recognition that SHH, one of the mammalian homologs of Drosophila HH, has been associated with prostate development (7) the present inventors have determined whether Perlecan might function in prostate cancer and whether human Perlecan associates with SHH signaling.

Tissue culture. LNCaP, PC3 and DU-145 cell lines were obtained from ATCC and grown under standard conditions. PC3-I and PC3-NI were derived at, and obtained from, the Medical College of Wisconsin. LN4 and Pro4 lines were obtained from M.D. Anderson Cancer Center, Houston. All primary prostate tumors were obtained using approved protocols with informed consent on the part of the subjects.

Real Time PCR on cell line RNA samples. Total RNA purified from cell lines using Trizol, digested with DNAse (Invitrogen), and analyzed using the SYBER Green system according to manufacturers protocols (Applied Biosystems) on an ABI Prism 7700 machine. Each sample was run in triplicate at three different concentrations. Primers were designed using Primer Express software and are available upon request.

Tissue microarray and immunohistochemistry. Upon institutional review board approval, a tissue microarray was prepared from 288 radical prostatectomy cases present at the Medical College of Wisconsin. A second tissue microarray was prepared from samples collected under approved protocols at the University of Pittsburgh Medical Center. Tissue microarrays had about 0.6 mm cores as arrayed and 5 μm sections were processed. Benign tissue, high-grade prostatic intraepithelial neoplasia or invasive tumor tissue were identified by Cytokeratin (CK903 Ab, DAKO) staining. For a portion of the tissue microarray anonymous de-identified pathologic and outcomes data were available. Individual cores were examined as duplicates and staining correlated using Chi-squared, Fisher's Exact or two-tailed ANOVA analyses.

Immunocytochemistry on cell lines was carried out using with anti-BrdU (Research Diagnostics or Becton-Dickinson) and HRP-conjugated secondary antibodies (Boehringer Mannheim) using standard techniques. For microarray samples, a common antigen retrieval procedure was carried out. Slides were processed for Perlecan and developed with HRP conjugated secondary antibodies and DAB substrate.

siRNA Perlecan inhibition. Purified and desalted siRNAs were purchased from Dharmacon, Inc. as double stranded 21 basepair oligonucleotides. The Perlecan siRNA sequence chosen was 5′ AAGGAGCUGGAUGGCUGGGUU 3′ (SEQ ID NO.: 1). The control siRNA purchased was: 5′ AACGUACGCGGAAUACAACGA 3′ (SEQ ID NO.: 2). SiRNA transfections (e.g., about 0.2 μM) were with Oligofectamine (Invitrogen) as described by the manufacturer and effects measured after about 70 hours.

Protein extracts, Western blotting and immunoprecipitations. Normal and tumor tissue from the same patients were obtained as described below following approved protocols. Sections were assessed pathologically by a urologic pathologist to determine areas of normal and tumor tissue. Samples were microdissected and total protein isolated. Total or extracellular matrix proteins were also isolated from cell lines grown under normal or serum starved conditions. Proteins were run on about a 4-20% non-denaturing gradient gel, blotted and probed for Perlecan (Chemicon) or GAPDH (Research Diagnostics) as a loading control. Equal amounts of protein were immunoprecipitated with an anti-Perlecan or unrelated control antibody, the resulting complex run on denaturing SDS-PAGE, and the presence of SHH verified by immunoblotting.

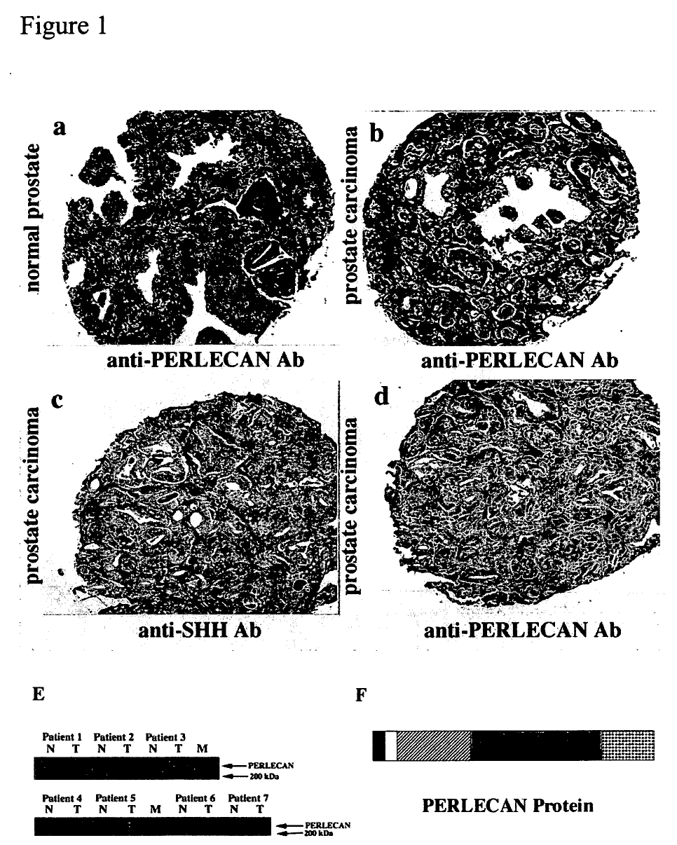

To examine the role of Perlecan in human prostate cancer the presence of Perlecan was determined in the prostate. It was found that Perlecan was elevated in prostate cancer tumors versus normal tissue. Next, immunolabeling of tissue microarrays containing prostate tumors was performed and demonstrated that Perlecan, a secreted protein, is present in the lumens of about 54% of malignant prostate cancer glands, but not in normal glands (FIGS. 1A-B, Table 1).

Briefly, FIGS. 1A to 1F demonstrate the Perlecan expression in human prostate tumors. Immunohistochemistry of Perlecan protein in FIG. 1A, normal prostate and FIG. 1B, prostate tumor. Perlecan is present as a secreted protein in the tumor gland lumens. The residual normal gland lumens in the left panel that fail to demonstrate intraluminal Perlecan. Comparison of FIG. 1C, SHH and FIG. 1D Perlecan localization in consecutive sections of prostate carcinoma. Both are secreted proteins and are present in tumor lumens. Examples of co-localization in gland lumens are highlighted (e.g., red asterisks, 2:00 position). A significant association between Perlecan and SHH staining was noted in prostate cancers (Table 1). FIG. 1E is a Western blot of protein from normal (N) and tumor (T) portions of specimens from individual patients. Labeled arrows indicate size marker (lane M) and Perlecan protein band. FIG. 1F, is a schematic of the human Perlecan protein structure. Amino terminus is to the left. The five domains of Perlecan are indicated by different patterns. Total protein length about 4370 amino acids. All histologic images were photographed at 400× magnification.

TABLE 1

| Perlecan | | Perlecan | Perlecan | |
|---|---|---|---|---|
| staining | | negative | positive | |
| Histology | Tumor | 170 | 203 | |
| | Normal | 211 | 31 | p < 0.00005 |
| | Tumor | 170 | 203 | |
| | HGPIN | 46 | 7 | p < 0.00005 |
| Clinical stage | cT2 | 11 | 12 | |
| | cT3/4 | 2 | 3 | N.S. |
| Tumor Grade | Gleason 6 | 26 | 5 | |
| | Gleason 7, | 38 | 23 | p = 0.0335 |
| | 8, 9 | | | |
| Pathologic | pT1-pT2 | 35 | 17 | |
| stage | pT3 | 29 | 11 | N.S. |
| Nodal status | pN0 | 18 | 23 | |
| | pN1 | 0 | 1 | N.S. |
| Outcomes | PSA | 4 | 5 | |
| | Recurrence | | | |
| | No PSA | 13 | 22 | N.S. |
| | recurrence | | | |
| Vital status | Alive | 26 | 36 | |
| | Dead | 2 | 5 | N.S. |
| | SHH negative | 69 | 63 | |
| | SHH positive | 10 | 58 | p < 0.00005 |

| | | | mean % of | |
|---|---|---|---|---|
| Ki-67 | sample | number of | Ki-67 | Two-tailed |
| staining | staining | samples | positivity | ANOVA |
| with | PERLECAN | 143 | 6.71 | p = 0.0478 |
| PERLECAN | positive | | | |
| | PERLECAN | 214 | 5.02 | |
| | negative | | | |

* Tumor grade is presented as Gleason score.
Pathologic staging uses the American Joint Commission on Cancer (AJCC) 2002 tumor staging criteria.
HGPIN = high grade prostatic intraepithelial neoplasia.

More detailed analysis of prostate cancer, the precancerous lesion high grade prostatic intraepithelial neoplasia (HGPIN), and benign prostate tissue from 600 patients was performed for Perlecan and was correlated with results obtained for the proliferation marker Ki-67 (PCNA). There was a significant increase in Perlecan levels in invasive tumors when compared to either benign prostate tissue or HGPIN. A subset of the patients had more detailed clinical, pathologic, and outcomes data available for analysis. In these patients, Perlecan expression was associated with more aggressive tumors, as evidenced by their higher Gleason score (e.g., Gleason score 7, 8, 9 versus Gleason score 4, 5, 6 tumors). Perlecan expression was significantly associated with increased prostate cancer cell proliferation, as demonstrated by Ki-67 staining, but not pathologic stage. Since Perlecan has been implicated in HH signaling in Drosophila, and a role for SHH has been described during prostate development (7), therefore, the staining patterns for Perlecan and SHH were compared in sequential tissue microarray sections. Co-localization of Perlecan and SHH staining was noted in a significant number of tumors, (FIGS. 1C-D, Table 1).

To extend the evaluation of Perlecan in patient samples Western analyses of matched prostate cancer and normal prostate tissues taken from the same patients (FIG. 1E) were examined. Similar to the percentage of tissue microarray tumor samples with high levels of luminal Perlecan protein, Perlecan protein was significantly increased in three out of seven matched patient tumor samples.

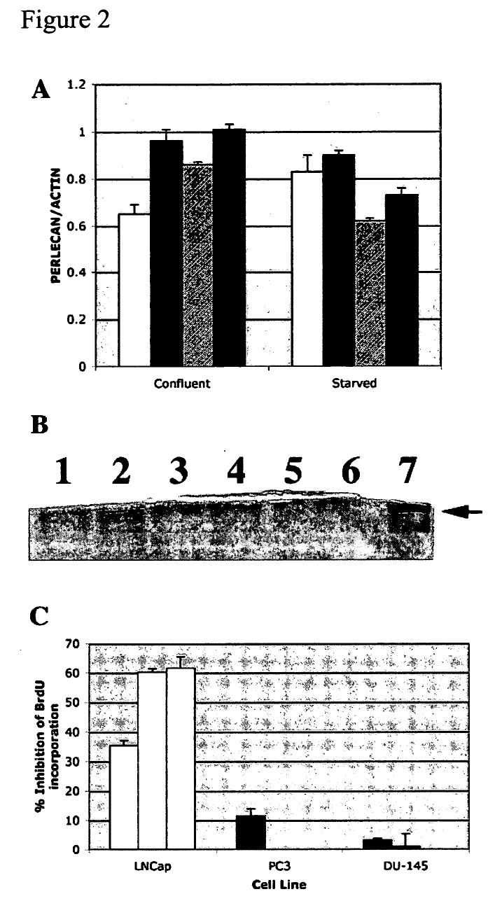

To further define the relationship of Perlecan expression with the development of invasive prostate cancer Real Time-PCR was used to evaluate Perlecan levels in human PC3 prostate cancer cell sublines demonstrating high or low invasive potential (PC3-I, PC3-NI) after serial passage through the Matrigel extracellular matrix invasion assay (8). Paired metastatic and non-metastatic cell lines (e.g., LN4, Pro4) were also analyzed as was an androgen sensitive cell line (LNCaP) derived from a prostate cancer lymph node metastasis (9). Perlecan mRNA was increased approximately 1.5 fold in confluent cultures of the high-invasive PC3 subline when compared to the low invasive subline, correlating with the induction of Perlecan protein seen in invasive tumors by immunohistochemistry. In addition, there was a 1.2 fold increase of Perlecan mRNA in confluent cultures of the metastatic LN4 cell line (FIG. 2A) when compared to the non-metastatic Pro4 cells. The LNCaP cell line had a Perlecan/ACTIN ratio similar to the invasive PC3-I cell line. The induction of Perlecan in the PC3-I versus PC3-NI cell lines was independent of cell density as measured by cell growth curves (data not shown).

Briefly, FIGS. 2A to 2C are graphs that show Perlecan expression and functional analysis in cell lines. FIG. 2A is a graph of a real time PCR of Perlecan in cell lines grown under normal or serum starved conditions. Non-invasive (PC3-NI, black column), Invasive (PC3-I, light grey column) grown under normal conditions or serum starvation (PC3-NI, dark grey column, PC3-I, white column), non-metastatic (Pro 4, striped dark grey column) and metastatic (LN4, striped light grey column). Error bars indicate standard deviation. FIG. 2B is an image showing that Perlecan protein from equal amounts of (lane 1) extracellular matrix extract of serum starved PC3-I cells, (lane 2) total protein from serum starved PC3-I cells, (lane 3) extracellular matrix extract of PC3-I cells, (lane 4) total protein of PC3-I cells, (lane 5) extracellular matrix extract of PC3-NI cells, (lane 6) total protein of PC3-NI and (lane 7) size marker (arrow indicates 200 kDa). FIG. 2C shows the effect of Perlecan siRNA on BrdU incorporation in the LNCaP (white bars), PC3 (grey bar) and DU-145 (black bars) cell lines. Error bars indicate standard deviation.

Heparan sulfate proteoglycans such as Perlecan have been shown to bind growth factors and may act as reservoirs or co-receptors for growth factors (6), thus amplifying their function under growth factor limiting conditions such as serum starvation. Previous studies have shown that the most prominent effects of Perlecan on cell growth are seen in serum starvation conditions (10, 11). To evaluate this potential role in prostate cancer the presence of Perlecan protein (FIG. 2B) was assayed in extracellular matrix extracts and total protein extracts from PC3-I and PC3-NI cells grown under normal and serum starved conditions. Perlecan expression increased under serum starvation conditions in both cell lines.

With reference to FIG. 2C, the graph relates the function of Perlecan in prostate cancer was tested in prostate cancer cell lines derived from lymph node (LNCaP) and bone (PC3 and DU-145) metastasis. A specific 21 nucleotide small interference RNA (siRNA) directed at Perlecan was used to inhibit gene function. Comparison of the results of transfecting Perlecan siRNA to those of a control siRNA revealed that interference with Perlecan function produced a decrease in BrdU incorporation in both the androgen sensitive cell line LNCaP (up to about 60%) and the androgen insensitive cell line PC3 (about 10%). Again with reference to FIG. 2C, little to no effect of Perlecan inhibition on BrdU incorporation was detected in the androgen insensitive cell line DU-145.

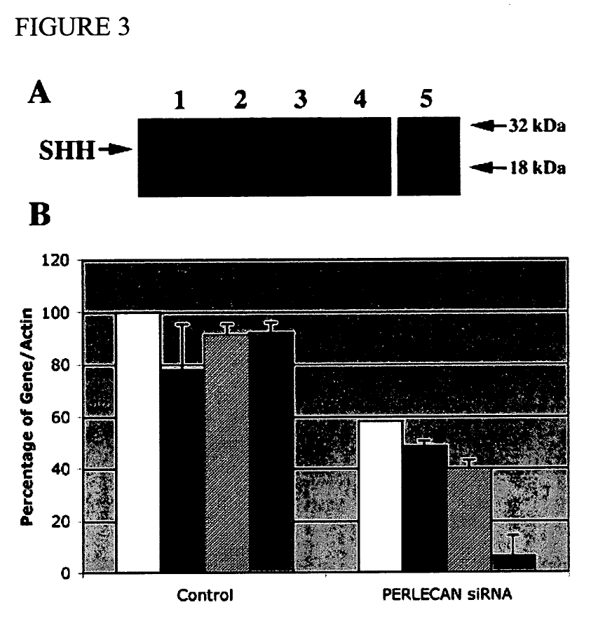

Briefly, FIGS. 3A and 3B demonstrate the interaction between the Perlecan and the SHH-GLI1 pathway. FIG. 3A is an image of a gel showing the co-immunoprecipitation of SHH with anti-Perlecan antibody and probed with anti-SHH. Extracts from (lane 1) extracellular matrix extract of serum starved PC3-I cells, (lane 2) total protein from serum starved PC3-I cells, (lane 3) extracellular matrix extract of PC3-I cells, (lane 4) extracellular matrix extract of PC3-NI cells and (lane 5) extracellular matrix extract of PC3-I cells precipitated with a control antibody. Placement of size markers is indicated. FIG. 3B shows the expression of Perlecan (black columns), PTCH (striped columns) and GLI1 (grey columns) normalized to 13-ACTIN levels in LNCaP cells treated with Perlecan siRNA and controls. Percent BrdU incorporation (white columns), normalized to control levels. Error bars indicate standard deviation.

The observed co-localization of SHH and Perlecan by immunohistochemistry, and the correlation of Perlecan with cellular proliferation rate raised the possibility that Perlecan may exert its effect on SHH signaling by binding to SHH in prostate cancers, as it does in the fly brain (4). With reference to FIG. 3A a graph to test for a direct interaction between Perlecan and SHH co-immunoprecipitation studies were performed from human PC3 prostate cancer cells. The 19 kDa mature SHH protein was identified by Western blotting in all protein extracts precipitated with anti-Perlecan antibodies but not from extracts precipitated with control antibodies.

To investigate whether human Perlecan can affect SHH signaling the expression of PTCH1 and GLI1 in LNCaP cells treated with Perlecan siRNA was determined. PTCH1 and GLI1 are targets of the active SHH-GLI pathway (12). Treated LNCaP cells showed about a 40% decrease in Perlecan expression, about a 60% decrease in the level of PTCH1 expression, and about a 90% decrease in GLI1 expression compared to controls, as well as about a 40% decrease in cellular proliferation (FIG. 3B). Thus, this result correlates the presence of PERLECAN with SHH-GLI pathway activity.

As shown herein, the interference with Perlecan function demonstrates that this proteoglycan is required for the growth of prostate cancer cells, extending its previously described roles in melanoma, colon, and lung cancer (11, 13, 14). High levels of Perlecan protein correlate significantly with aggressive, highly proliferating prostate tumors in the tissue microarrays and are also observed in tumor versus normal samples from individual patients. This correlation extends to prostate cancer cell lines, in which higher levels of Perlecan message and protein were detected in invasive or metastatic cell lines. Furthermore, when cell lines were grown under serum starvation conditions Perlecan mRNA and protein increased, supporting the hypothesis that Perlecan is needed to concentrate or stimulate binding of growth factors, such as FGFs and HHs, to their receptors (6). Thus, Perlecan may increase the growth of starved cancer cells in rapidly spreading tumors by amplifying their sensitivity and response to growth factor signaling. The possibility that mutations in Perlecan could explain the increased incidence of brain and prostate familial tumors and be the gene mutated in CABP is intriguing, as the SHH-GLI pathway also has been implicated in brain tumors of glial origin (15). However, since Perlecan also interacts with or affects many signaling pathways (6), it is not yet clear which signaling pathways are the critical Perlecan-dependent events in prostate cancer growth. Recent results (e.g., Savore, et al.) suggest that Perlecan may regulate the activity of multiple growth factors in prostate cancer cells.

The decrease in SHH signaling in cells lacking Perlecan as well as the detection of a SHH-Perlecan complex extends to humans previous results from the present inventors showing that Perlecan modulates HH signaling in flies. Perlecan may also be involved in other developmental processes that require SHH signaling, and dysregulated Perlecan and/or growth factor expression may result in disease. Heparan sulfate proteoglycans have been implicated in exostoses, a disease of overexuberant bone formation (16). Heparan sulfate proteoglycans have also been associated with signaling by HHs, BMPs and WNTs (17), all of which have been implicated in bone formation (18). Thus, changes in Perlecan in prostate cancer could alter the function of one or multiple signaling pathways, resulting in the osteoblastic lesions characteristic of metastatic states.

The present invention includes a method of diagnosing cancer and pre-cancer pathology in a subject by obtaining tissue sample from the subject and analyzing the level of expression of Perlecan, Patched or Smoothened, for elevated Perlecan, Patched or Smoothened expression indicative of cancer or pre-cancer. The sample may be from a verity of areas throughout the body of the subject and in a verity of types (e.g., tissue sample, serum sample, liquid sample extracted sample and the like). In one embodiment, the sample is a prostate tissue sample, however a serum or semen sample are also contemplated. In the analyzing of the sample using techniques common to persons of ordinary skill in the art, including light microscopy, electron microscopy, immunostaining and the like.

The present invention may also be used to treat individual with prostate cancer or at risk of developing prostate cancer, by identifying an individual with prostate cancer or at risk of developing prostate cancer, administering a dose of a Perlecan specific siRNA to the individual in an amount that is effective to inhibit the expression of Perlecan and monitoring the expression of Perlecan in the individual, wherein inhibiting the expression of Perlecan inhibits the proliferation of prostate cancer cells, thereby treating the individual.

The present invention may be administration in many different manners including oral, transdermal, intravenous, intraperitoneal, and implanted and be packaged into a capsule, caplet, softgel, gelcap, suppository, film, granule, gum, insert, pastille, pellet, troche, lozenge, disk, poultice or wafer. In addition the effective dose may be from about 5 mg/kg to about 25 mg/kg. However, other doses may be used depending on the circumstances of the situation (e.g., about 1 mg/kg to about 5 mg/kg, about 25 mg/kg to about 50 mg/kg, and greater than 50 mg/kg).

The present invention also includes a system for identifying inhibitors of Perlecan, by adding one or more molecules suspected of modifying Perlecan expression to a cell that expressed Perlecan and analyzing the level of expression of Perlecan in the cells, for an elevated level of expression indicative of cancer or pre-cancer. The one or more molecules by be nucleic acids, peptides, small molecules, proteins, lipids, carbohydrates, entire cells, cellular extracts, homogenized tissue or cells, and combinations and mixtures thereof. The level of expression of Prelecan in the cells can be compared to a non-treated cell control, standard control expression levels expression over time, or a combination thereof. The cell are selected from prostate cancer, small cell lung cancer, stomach cancer, pancreatic cancer, malignant melanoma, basal cell carcinoma, colon cancer and glioma cells.

Finally, Perlecan, as a secreted protein, may prove to be a useful biomarker for prostate cancer as well as a marker of either the risk or detection of tumor metastasis to bone since it can be easily detected in urine or serum samples, respectively. Use of Perlecan as a drug target might also be advantageous, either alone or in combination with the current hormone blocking therapies as a method of inhibiting prostate cancer and blocking bone metastasis ands/or its “bone pain” associated morbidity.

Additionally, the use of Perlecan as a drug may also be advantageous, either alone or in combination with other active agents. The present invention also includes a pharmaceutical composition including a pharmaceutically effective amount of a Perlecan specific siRNA, wherein the Perlecan specific siRNA has SEQ ID NO.: 1 and one or more carriers.

The pharmaceutical composition may be in the form of a capsule, a suppository, a gel cap, a softgel, a lozenge, a sachet or even a fast dissolving wafer. As used herein the term “carrier” is used to describe a substance, whether biodegradable or not, that is physiologically acceptable for human or animal use and may be pharmacologically active or inactive.

The pharmaceutical composition may also be provided in a variety of dosage forms, e.g., solution, suspension, cream, ointment, lotion, capsule, caplet, softgel, gelcap, suppository, enema, elixir, syrup, emulsion, film, granule, gum, insert, jelly, foam, paste, pastille, pellet, spray, troche, lozenge, disk, magma, poultice, or wafer and the like.

For gelcap preparations, the pharmaceutical formulation may include oils, e.g.: (1) fixed oils, such as peanut oil, sesame oil, cottonseed oil, corn oil and olive oil; (2) fatty acids, such as oleic acid, stearic acid and isostearic acid; and fatty acid esters, such as ethyl oleate, isopropyl myristate, fatty acid glycerides and acetylated fatty acid glycerides; (3) alcohols, such as ethanol, isopropanol, hexadecyl alcohol, glycerol and propylene glycol; (4) glycerol ketals, such as 2,2-dimethyl-1,3-dioxolane-4-methanol; (5) ethers, such as poly(ethylene glycol) 450; (6) petroleum hydrocarbons, such as mineral oil and petrolatum; and (7) water, or with mixtures thereof; with or without the addition of a pharmaceutically suitable surfactant, suspending agent or emulsifying agent.

For oral, buccal, and sublingual administration, the pharmaceutical composition of the invention may be administered as either solutions (e.g., in saline or other physiologic compatible solution and/or buffer) or suspensions in the form of gelcaps, caplets, tablets, capsules or powders (e.g., sterile lyophilized, vacuum dried and the like). For rectal administration, the compounds of the invention may be administered in the form of suppositories, ointments, enemas, tablets and creams for release of compound in the intestines, sigmoid flexure and/or rectum. For example, when making a suppository a beeswax/glycerol composition may be used to form a body meltable suppository for transrectal or transurethral delivery. The composition may be administered in oral, transdermal, intravenous, intraperitoneal, pulmonary, rectal, ocular, subcutaneous, intramuscular, transdermal, topical, intraosseal, epidural, dural, intranasal, and implanted.

The pharmaceutical composition may also be administered as a liquid suspension or solution using a sterile liquid, e.g., oil, water, an alcohol, or mixtures thereof, with or without the addition of a pharmaceutically suitable surfactant, suspending agent, or emulsifying agent for oral or parenteral administration. For liquid preparations, the pharmaceutical composition can be formulated suitably with water, oils, for example, fixed oils, such as peanut oil, sesame oil, cottonseed oil, corn oil and olive oil; fatty acids, such as oleic acid, stearic acid and isotearic acid; and fatty acid esters, such as ethyl oleate, isopropyl myristate, fatty acid glycerides and acetylated fatty acid glycerides; with alcohols, such as ethanol, isopropanol, hexadecyl alcohol, glycerol and propylene glycol; with glycerol ketals, such as 2,2-dimethyl-1,3-dioxolane-4-methanol; with ethers, such as poly(ethyleneglycol) 450, with petroleum hydrocarbons, such as mineral oil and petrolatum; with water, or with mixtures thereof; with or without the addition of a pharmaceutically suitable surfactant, suspending agent or emulsifying agent.

The pharmaceutical composition and/or the solid carrier particles can be coated with one or more enteric coatings, seal coatings, film coatings, barrier coatings, compress coatings, fast disintegrating coatings, or enzyme degradable coatings. Multiple coatings may be applied for desired performance. Further, some actives may be provided for immediate release, pulsatile release, controlled release, extended release, delayed release, targeted release, synchronized release, or targeted delayed release. For release/absorption control, solid carriers can be made of various component types and levels or thicknesses of coats, with or without an active ingredient. Such diverse solid carriers can be blended in a dosage form to achieve a desired performance. The compositions may be formulated for oral, nasal, buccal, ocular, urethral, transmucosal, vaginal, topical or rectal delivery.

When formulated as a capsule, the capsule can be a hard or soft gelatin capsule, a starch capsule, or a cellulosic capsule. Although not limited to capsules, such dosage forms may be further coated with, for example, a seal coating, an enteric coating, an extended release coating, or a targeted delayed release coating.

Dosage forms of the compositions of the present invention can also be formulated as enteric coated delayed release oral dosage forms, i.e., as an oral dosage form of a pharmaceutical composition as described herein that uses an enteric coating to effect release in the lower gastrointestinal tract. The enteric coated dosage form may be a compressed or molded or extruded tablet/mold (coated or uncoated) containing granules, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated.

The coating may also contain a plasticizer and possibly other coating excipients such as colorants, talc, and/or magnesium stearate, which are well known in the art. Suitable plasticizers include: triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain about 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the lower intestinal tract is reached.

Immediate release coating of solid carriers is commonly used to improve product elegance as well as for a moisture barrier, and taste and odor masking. Rapid breakdown of the film in gastric media is important, leading to effective disintegration and dissolution. Eudragit RD100 (Rohm) is an example of such a coating. It is a combination of a water insoluble cationic methacrylate copolymer with a water-soluble cellulose ether. In powder form, it is readily dispensible into an easily sprayable suspension that dries to leave a smooth film. Such films rapidly disintegrate in aqueous media at a rate that is independent of pH and film thickness.

In addition to the active agent a variety of inactive agents may be added to the pharmaceutical formulation. In-Actives agents include but not limited to the following. Sugar Spheres: Sugar spheres are used as inert cores in capsule and tablet formulations particularly multiparticulate sustained release formulations and are provided in amounts sufficient to accept the active ingredient for extended release, e.g., phenlyepherine. Sugar spheres are generally of relatively uniform diameter and contain 62.5%-91.5% sucrose with the remainder being starch.

Pharmaceutical Glaze: (about 4.5 mg) Shellac is a natural occurring material, consisting of a complex mixture of constituents. The main component of shellac (˜95%) is a resin that upon mild basic hydrolysis gives a mixture of compounds of high plasticity. Shellac is used extensively in the pharmaceutical industry as a film coating agent for beads and tablets.

The substrate of the compositions of the present invention may be a powder or a multiparticulate, such as a granule, a pellet, a bead, a spherule, a beadlet, a microcapsule, a millisphere, a nanocapsule, a nanosphere, a microsphere, a platelet, a minitablet, a tablet or a capsule. A powder constitutes a finely divided (milled, micronized, nanosized, precipitated) form of an active ingredient or additive molecular aggregates or a compound aggregate of multiple components or a physical mixture of aggregates of an active ingredient and/or additives. Such substrates may be formed of various materials known in the art, such as, for example: sugars, such as lactose, sucrose or dextrose; polysaccharides, such as maltodextrin or dextrates; starches; cellulosics, such as microcrystalline cellulose or microcrystalline cellulose/sodium carboxymethyl cellulose; inorganics, such as dicalcium phosphate, hydroxyapitite, tricalcium phosphate, talc, or titania; and polyols, such as mannitol, xylitol, sorbitol or cyclodextrin.

It should be emphasized that a substrate need not be a solid material, although often it will be a solid. For example, the encapsulation coat on the substrate may act as a solid “shell” surrounding and encapsulating a liquid, semi-liquid, powder or other substrate material. Such substrates are also within the scope of the present invention, as it is ultimately the carrier, of which the substrate is a part, which must be a solid.

The solid pharmaceutical compositions of the present invention may include optionally one or more additives, sometimes referred to as additives. The excipients may be contained in an encapsulation coat in compositions, which include an encapsulation coat, or can be part of the solid carrier, such as coated to an encapsulation coat, or contained within the components forming the solid carrier. Alternatively, the excipients can be contained in the pharmaceutical composition but not part of the solid carrier itself.

Suitable excipients are those used commonly to facilitate the processes involving the preparation of the solid carrier, the encapsulation coating, or the pharmaceutical dosage form. These processes include agglomeration, air suspension chilling, air suspension drying, balling, coacervation, comminution, compression, pelletization, cryopelletization, extrusion, granulation, homogenization, inclusion complexation, lyophilization, nanoencapsulation, melting, mixing, molding, pan coating, solvent dehydration, sonication, spheronization, spray chilling, spray congealing, spray drying, or other processes known in the art. The excipients may also be pre-coated or encapsulated, as are well known in the art.

The pharmaceutical compositions of the present invention may include optionally one or more solubilizers, i.e., additives to increase the solubility of the pharmaceutical active ingredient or other composition components in the solid carrier. Mixtures of solubilizers are also within the scope of the invention and are readily available from standard commercial sources.

The amount of solubilizer that may be included in compositions of the present invention is not particularly limited. Of course, when such compositions are administered to a patient, the amount of a given solubilizer is limited to a bioacceptable amount, which is readily determined by one of skill in the art. In some circumstances, it may be advantageous to include amounts of solubilizers far in excess of bioacceptable amounts, for example, to maximize the concentration of active ingredient, with excess solubilizer removed prior to providing the composition to a patient using conventional techniques, such as distillation or evaporation.

Other additives conventionally used in pharmaceutical compositions may be included, which are well known in the art. Such additives include, e.g.,: anti-adherents (anti-sticking agents, glidants, flow promoters, lubricants) such as talc, magnesium stearate, fumed silica), micronized silica, polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate.

Other additives include, binders (adhesives), i.e., agents that impart cohesive properties to powdered materials through particle-particle bonding, such as matrix binders (dry starch, dry sugars), film binders (PVP, starch paste, celluloses, bentonite and sucrose), and chemical binders (polymeric cellulose derivatives, such as carboxy methyl cellulose, HPC and HPMC; sugar syrups; corn syrup; water soluble polysaccharides such as acacia, tragacanth, guar and alginates; gelatin; gelatin hydrolysate; agar; sucrose; dextrose; and non-cellulosic binders, such as PVP, PEG, vinyl pyrrolidone copolymers, pregelatinized starch, sorbitol, and glucose).

For certain actives it may be useful to provide buffering agents (or bufferants), where the acid is a pharmaceutically acceptable acid, such as hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid, and where the base is a pharmaceutically acceptable base, such as an amino acid, an amino acid ester, ammonium hydroxide, potassium hydroxide, sodium hydroxide, sodium hydrogen carbonate; aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, or a salt of a pharmaceutically acceptable cation and acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, and uric acid.

In some formulations additives may also include: chelating agents (such as EDTA and EDTA salts); colorants or opaquants (such as titanium dioxide, food dyes, lakes, natural vegetable colorants, iron oxides, silicates, sulfates, magnesium hydroxide and aluminum hydroxide); coolants (e.g., trichloroethane, trichloroethylene, dichloromethane, fluorotrichloromethane); cryoprotectants (such as trehelose, phosphates, citric acid, tartaric acid, gelatin, dextran and mannitol); and diluents or fillers (such as lactose, mannitol, talc, magnesium stearate, sodium chloride, potassium chloride, citric acid, spray-dried lactose, hydrolyzed starches, directly compressible starch, microcrystalline cellulose, cellulosics, sorbitol, sucrose, sucrose-based materials, calcium sulfate, dibasic calcium phosphate and dextrose).

Yet other additives may include disintegrants or super disintegrants; hydrogen bonding agents, such as magnesium oxide; flavorants or desensitizers; ion-exchange resins, such as styrene/divinyl benzene copolymers, and quaternary ammonium compounds; plasticizers, such as polyethylene glycol, citrate esters (e.g., triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl seccate; and preservatives, such as ascorbic acid, boric acid, sorbic acid, benzoic acid, and salts thereof, parabens, phenols, benzyl alcohol, and quaternary ammonium compounds.

The compositions of the present invention can be prepared by a variety of processes to apply an encapsulation coat onto a substrate or to form a substrate-free solid carrier such as a multiparticulate or a powder. The most commonly used coating and pelletization processes include: balling, spheronization, extrusion, spray congealing, spray drying, pan coating, fluidized bed coating, melt extrusion, crystallization, cryopelletization, nanoencapsulation, coacervation, etc. One skilled in the art will recognize that appropriate additives may also be introduced to the composition or during the processes to facilitate the preparation of the solid carrier or the dosage forms, depending on the need of the individual process.

A pelletization process typically involves preparing a molten solution of the composition of the solid carrier or a dispersion of the composition of the solid carrier solubilized or suspended in an aqueous medium, an organic solvent, a supercritical fluid, or a mixture thereof. Such solution or dispersion is then passed through a certain opening to achieve the desired shape, size, and other properties. Similarly, appropriate drying processes may be used to control the level of the residual dispersing medium, if necessary. The processes described above, the combination of the processes, or the modification of the processes are well know in the art. Some of the processes are briefly described herein for reference.

In addition, the pharmaceutical composition of the present invention may include an ion exchange substrate. The loading of the drug on the resin particles can be from about 1-90 percent by weight, although about 15-50 percent is the normal practical range. A wide range of cationic (for the basic drugs) or anionic (for the acidic drugs) exchange resins can be used to form the drug resin complex, particle sizes normally ranging from about 75 to 1000 um. Examples include, Amberlite IR-120, a cationic exchange resin consisting of about 20-30 mesh (about 590-840 um) spherical particles as a model large particle resin and Amberlite XE-69, which is 100-200 mesh fractured resin particles of Amberlite IR-120, as a model small particle resin. The parent resin of IR-120 and XE-69 is described by the manufacturer as gel-type divinylbenzene sulfonic acid cation exchange resin which swells in water with a pH range of 0-14. Other suitable ion exchange resin candidates include synthetic ion exchange resins with different polymeric matrices (e.g., methacrylic, acrylic, phenol formaldehyde), ion exchange agents with cellulosic or dextran polymer matrices, and inorganic ion exchange matrices. The resins should not have inherent pharmacological or toxic properties.

Adsorption of the drug onto the ion exchange resin particles to form the active agent-resin complex is a well-known technique as shown in U.S. Pat. No. 2,990,332 (relevant portions incorporated herein by reference) and demonstrated in the examples hereinbelow. In general, the drug is mixed with an aqueous suspension of the resin and the complex is then dried. Adsorption of drug onto the resin is detected by a change in the pH of the reaction medium.

Particle coating. Another example of a coated particle for use with the present invention is disclosed in U.S. Pat. No. 4,221,778, in which a selective, prolonged continuous release of pharmacologically active drugs, under conditions such as those encountered in the gastrointestinal tract, is achieved by the application of a diffusion barrier coating to an ion exchange drug-resin complex particle which has been treated with a solvating agent. Another prolonged release formulations from coated drugs of the Raghunathan type can be prepared under circumstances wherein a component of the formulation contains a second ionic substance (e.g. a combination drug, a dye, a dispersing agent or the like) bearing the same ionic charge as the drug on the drug-resin complex by employing the second ionic substance in the ion form of an exchange resin complex. The manufacture of a formulation of any drug for liquid dosage usage requires that the final formulation have the drug dissolved or suspended in a liquid that is pleasing to the eye and taste, possess extended shelf-life stability, and exhibit no change in active drug dosage level over a period of time. Thus, to prepare a liquid formulation of any type drug, including one of the Raghunathan type, it is necessary to employ extenders such as water or syrup, and to add flavors, sweeteners, thickening agents, dyes, and the like. To control the dissolution profile of the formulation versus the dissolution profile of the same drug in water, the coated particles may also be included in the presence of ionic substances bearing the same ionic charge as the sustained release drug present in the formulation as a coated drug-resin complex. The presence of ionic substances of opposite charge in the final solution, do not have an effect on the expected dissolution rate and improve the release profile. Resins suitable for binding the second ionic component may be any of those previously disclosed in the Raghunathan patent. The second ionic material need not be coated with the water-permeable diffusion barrier coating.

The water-permeable, diffusion barrier coating material may be a conventional synthetic or natural film-forming materials with diffusion barrier properties and with no inherent pharmacological or toxic properties. For example, ethylcellulose (U.S.P. grade), a water insoluble film-forming agent was used as the model diffusion barrier membrane material in the illustrative examples. A plasticizer, Durkex 500 vegetable oil, was used to improve the film-forming charateristics of ethylcellulose. The amount of ethylcellulose film coating used depends on the degree of drug release prolongation desired.

Conventional coating solvents (such as ethanol, or a methylene chloride/acetone mixture, or coating emulsions) and coating procedures can be employed to coat the particles. In the illustrative examples, coatings were carried out by using a Wurster coating apparatus. Techniques of fluid bed spray coating are taught, for example, in U.S. Pat. Nos. 3,089,824; 3,117,027; and 3,253,944, relevant portions incorporated herein by reference. The coating may applied to the drug resin complex or may be applied to the resin before complexing with the drug.

In a broad sense, pellets are very much like granules and bead; the techniques for producing pellets may also produce granules, beads, etc. Pellets, granules or beads are formed with the aid of, e.g., a pelletizer, a spheronizer or an extruder. The pelletizer, spheronizer or extruder is able to form approximately spherical bodies from a mass of finely divided particles continuously, by a rolling or tumbling action on a flat or curved surface with the addition of a liquid.

Pelletizers are generally classified based on the angle of their axis as a horizontal drum or an inclined dish pelletizer. Rotary fluidized granulators may also be used for pelletization. A standard fluidized drier bowl may be replaced with a rotating plate as an air distributor. For granulation, a binder liquid is sprayed from via one or two binary nozzles located axially to the rotational movement of the powder bed. The granulation results in rounding of the granules to approximately spherical pellets. Such balling or agitation techniques are generally influenced by operating conditions, e.g., the bridging/binding liquid requirements, the residence time of the material in the pelletizer, the speed and angle of inclination of the pelletizer, the amount of material fed to the pelletizer and the choice and levels of binder, etc. Those skilled in the art may adjust readily such factors to produce a satisfactory product.

The choice of binder for a given application may also be determined readily by those skilled in the art. Generally, the binder must be capable of wetting the surfaces of the particle being pelletized or granulated. In general, binders must have sufficient wet strength to allow agglomerates to be handled and sufficient dry strength to make them suitable for their intended purposes. Each process, however, makes use of a different system of forces and may require a different agglomerate strength. The final selection of the binder is made generally based on the type of equipment used. Factors that affect the equipment and binder choices include: the size and size distribution of pellets, bulk density, strength and flow properties. Other factors that affect the performance of the pellets, which may be adjusted by one skilled in the art by the inclusion of additives, choice of equipment and processing conditions.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

1. Conlon, E. M., Goode, E. L., Gibbs, M., Stanford, J. L., Badzioch, M., Janer, M., Kolb, S., Hood, L., Ostrander, E. A., Jarvik, G. P., and Wijsman, E. M. Oligogenic segregation analysis of hereditary prostate cancer pedigrees: evidence for multiple loci affecting age at onset. Int J Cancer, 105: 630-635, 2003.
2. Gibbs, M., Stanford, J. L., Mclndoe, R. A., Jarvik, G. P., Kolb, S., Goode, E. L., Chakrabarti, L., Schuster, E. F., Buckley, V. A., Miller, E. L., Brandzel, S., Li, S., Hood, L., and Ostrander, E. A. Evidence for a rare prostate cancer-susceptibility locus at chromosome 1p36. Am J Hum Genet, 64: 776-787, 1999.
3. Datta, S. Control of proliferation activation in quiescent neuroblasts of the Drosophila central nervous system. Development, 121: 1173-1182, 1995.
4. Park, Y., Rangel, C., Reynolds, M. M., Caldwell, M. C., Johns, M., Nayak, M., Welsh, C. J., McDermott, S., and Datta, S. Drosophila perlecan modulates FGF and hedgehog signals to activate neural stem cell division. Dev Biol, 253: 247-257, 2003.
5. Dodge, G. R., Kovalszky, I., Chu, M. L., Hassell, J. R., McBride, O. W., Yi, H. F., and Iozzo, R. V. Heparan sulfate proteoglycan of human colon: partial molecular cloning, cellular expression, and mapping of the gene (HSPG2) to the short arm of human chromosome 1. Genomics, 10: 673-680, 1991.
6. Iozzo, R. V. and San Antonio, J. D. Heparan sulfate proteoglycans: heavy hitters in the angiogenesis arena. J. Clin. Invest., 108: 349-355, 2001.
7. Barnett, D. H., Huang, H. Y., Wu, X. R., Laciak, R., Shapiro, E., and Bushman, W. The human prostate expresses sonic hedgehog during fetal development. J Urol, 168: 2206-2210, 2002.
8. Lindholm, P. F., Bub, J., Kaul, S., Shidham, V. B., and Kajdacsy-Balla, A. The role of constitutive NF-kappaB activity in PC-3 human prostate cancer cell invasive behavior. Clin Exp Metastasis, 18: 471-479, 2000.
9. Horoszewicz, J. S., Leong, S. S., Chu, T. M., Wajsman, Z. L., Friedman, M., Papsidero, L., Kim, U., Chai, L. S., Kakati, S., Arya, S. K., and Sandberg, A. A. The LNCaP cell line—a new model for studies on human prostatic carcinoma. Prog Clin Biol Res, 37: 115-132, 1980.
10. Adatia, R., Albini, A., Carlone, S., Giunciuglio, D., Benelli, R., Santi, L., and Noonan, D. M. Suppression of invasive behavior of melanoma cells by stable expression of anti-sense perlecan cDNA. Annals of Oncology, 8: 1257-1261, 1997.
11. Sharma, B., Handler, M., Eichstetter, I., Whitelock, J. M., Nugent, M. A., and lozzo, R. V. Antisense targeting of perlecan blocks tumor growth and angiogenesis in vivo. J Clin Invest, 102: 1599-1608, 1998.
12. Lee, J., Platt, K. A., Censullo, P., and Ruiz i Altaba, A. Gli1 is a target of Sonic hedgehog that induces ventral neural tube development. Development, 124: 2537-2552, 1997.
13. Cohen, I. R., Murdoch, A. D., Naso, M. F., Marchetti, D., Berd, D., and Iozzo, R. V. Abnormal expression of perlecan proteoglycan in metastatic melanomas. Cancer Res, 54: 5771-5774, 1994.
14. Nackaerts, K., Verbeken, E., Deneffe, G., Vanderschueren, B., Demedts, M., and David, G. Heparan sulfate proteoglycan expression in human lung-cancer cells. Int J Cancer, 74: 335-345, 1997.
15. Dahmane, N., Sanchez, P., Gitton, Y., Palma, V., Sun, T., Beyna, M., Weiner, H., and Ruiz i Altaba, A. The Sonic Hedgehog-Gli pathway regulates dorsal brain growth and tumorigenesis. Development, 128: 5201-5212, 2001.
16. Hecht, J. T., Hall, C. R., Snuggs, M., Hayes, E., Haynes, R., and Cole, W. G. Heparan sulfate abnormalities in exostosis growth plates. Bone, 31: 199-204, 2002.
17. Christian, J. L. BMP, Wnt and Hedgehog signals: how far can they go? Curr Opin Cell Biol, 12: 244-249, 2000.
18. van der Horst, G., Farih-Sips, H., Lowik, C. W., and Karperien, M. Hedgehog stimulates only osteoblastic differentiation of undifferentiated KS483 cells. Bone, 33: 899-910, 2003.

What is claimed is:

1. A method of diagnosing cancer pathology in a subject comprising the steps of:
obtaining tissue sample from the subject; and
analyzing the level of expression of Perlecan, Patched or Smoothened, for Perlecan, Patched or Smoothened expression indicative of cancer or pre-cancer.

2. The method of claim 1, wherein the tissue sample comprises a prostate sample.

3. The method of claim 1, wherein the tissue sample comprises a sample suspected of prostatitis or prostate cancer.

4. The method of claim 1, wherein the tissue sample is prostate cancer exudate.

5. The method of claim 1, wherein the analysis is performed by light microscopy.

6. The method of claim 1, wherein the tissue sample is stained with haematoxylin and eosin.

7. The method of claim 1, wherein the analysis comprises electron microscopy.

8. The method of claim 1, wherein the analysis comprises immunostaining.

9. The method of claim 1, wherein the tissue sample is a serum or semen sample.

10. A method of treating an individual with prostate cancer or at risk of developing prostate cancer, comprising the steps of:
identifying an individual with prostate cancer or at risk of developing prostate cancer;
administering a dose of a Perlecan specific siRNA to the individual in an amount that is effective to inhibit the expression of Perlecan; and
monitoring the expression of Perlecan in the individual, wherein inhibiting the expression of Perlecan inhibits the proliferation of prostate cancer cells, thereby treating the individual.

11. The method of claim 10, wherein said administration is selected from the group consisting of oral, transdermal, intravenous, intraperitoneal, pulmonary, rectal, ocular, subcutaneous, intramuscular, transdermal, topical, intraosseal, epidural, dural, intranasal, and implanted.

12. The composition of claim 10, wherein the dose is packed into a vial, capsule, caplet, softgel, gelcap, suppository, film, granule, gum, insert, pastille, pellet, troche, lozenge, disk, poultice or wafer.

13. The method of claim 10, wherein the dose is from about 5 mg/kg to about 25 mg/kg.

14. The method of claim 10, wherein said individual is a human.

15. A method of reducing the risk of recurrence of prostate cancer in an individual, wherein said individual previously had been treated for prostate cancer, comprising the step of:
administering a dose of one or more Perlecan specific siRNA to the individual in an amount that is effective to inhibit the expression of Perlecan.

16. The method of claim 15, further comprising the step of periodically providing one or more Perlecan specific siRNA to the individual in an amount that is effective to inhibit the expression of Perlecan, thereby reducing the risk of recurrence of prostate cancer in the individual.

17. The method of claim 16, further comprising the step of:
monitoring the level of expression of Perlecan in the individual.

18. A system for identifying inhibitors of Perlecan, comprising the steps of:
adding one or more molecules suspected of modifying Perlecan expression to a cell that expressed Perlecan; and
analyzing the level of expression of Perlecan in the cells, for an elevated level of expression indicative of cancer or pre-cancer.

19. The system of claim 18, wherein the level of expression of Prelecan in the cells are compared to a non-treated cell control.

20. The system of claim 18, wherein the one or more molecules are selected from the group consisting of nucleic acids, peptides, small molecules, proteins, lipids, carbohydrates and combinations thereof.

21. The system of claim 18, wherein the expression of Perlecan is performed via a high-throughput, automated robot.

22. The system of claim 18, wherein the cells are selected from prostate cancer, small cell lung cancer, stomach cancer, pancreatic cancer, malignant melanoma, basal cell carcinoma, colon cancer and glioma cells.

23. A pharmaceutical composition comprising:
a pharmaceutically effective amount of a Perlecan specific siRNA, wherein the a Perlecan specific siRNA has (SEQ ID NO.: 1); and
one or more carriers.

24. The composition of claim 23, wherein the Perlecan specific siRNA is packaged into a vial, capsule, caplet, softgel, gelcap, suppository, film, granule, gum, insert, pastille, pellet, troche, lozenge, disk, poultice or wafer.

25. The composition of claim 23, wherein the pharmaceutically effective amount is from about 5 mg/kg to about 25 mg/kg.

26. The Perlecan specific siRNA having the sequence SEQ. ID No.:1.
